(19) United States
(12) Patent Application Publication
(10) Pub. No.: US 2020/0393334 A1
(43) Pub. Date: Dec. 17, 2020

(54) MONITORING SYSTEM AND CONTROL MODULE THEREOF

(71) Applicant: KAOHSIUNG VETERANS GENERAL HOSPITAL, KAOHSIUNG CITY (TW)

(72) Inventors: HSIEN-CHUNG YU, KAOHSIUNG CITY (TW); WEI-LING CHEN, KAOHSIUNG CITY (TW)

(21) Appl. No.: 16/669,757

(22) Filed: Oct. 31, 2019

(30) Foreign Application Priority Data
Jun. 14, 2019 (TW) 108120733

(51) Int. Cl. G01N 1/14; B08B 9/08

(52) U.S. Cl. CPC G01N 1/14; B08B 9/08

(57) ABSTRACT

A monitoring system and a control module thereof for monitoring a working fluid in a tank are disclosed. The monitoring system includes the tank, a sampling unit, a detecting agent tank, and a control module. The sampling unit is to obtain a sample of the working fluid from a sampling opening of the tank. The detecting agent tank is to inject a detecting agent to the sample. A detector of the control module is to detect the sample and input a detection result to a processing unit of the control module for subsequent processing. The control module can periodically obtain the sample through a timer. When the detection result exceeds a setting value, the processing unit will turn off the power switch of the working machine to stop the operation process and notify the operator to replace the working fluid.

### FIELD OF THE INVENTION

The present invention relates to a monitoring system and a control module thereof, and more particularly to a monitoring system and a control module thereof to obtain a sample of a working fluid in a tank automatically or periodically for determining whether the working fluid is usable.

### BACKGROUND OF THE INVENTION

The second point of the top ten medical hazards in 2017 is “Inadequate Cleaning of Complex Reusable Instruments Can Lead to Infections”.

For example, the disinfectant to clean endoscopes used in the hospitals is o-phthalaldehyde. Because endoscopes are reused, the cleaning of endoscopes is very important. Determining whether the disinfectant continues to be used will impact on the disinfection effect of the endoscopes. So far the way to determine whether the disinfectant continues to be used is to use a test paper. The principle of the detection is to use o-phthalaldehyde to react with sodium sulfite in the test paper to produce sodium hydroxide. When the concentration of o-phthalaldehyde is more than the minimum effective concentration (0.3%), the pH of the alkaline solution of sodium hydroxide will change the test paper to purple. Therefore, when the test paper is purple, the disinfectant can continue to be used. If the discoloration is uneven or the color of the test paper is not changed, the disinfectant must be replaced. Although this way can quickly determine whether the disinfectant is usable or not, it may cause errors due to different interpretations of different users. Therefore, it may affect the disinfection effect of the endoscopes.

Taiwan Patent Application No. 108110713 discloses a method for detecting disinfectant of medical equipment filed by the inventor, which detects the pH value of a sample of the disinfectant to determine whether the concentration of the disinfectant is acceptable. However, in actual implementation, because the number of endoscopes used in the hospitals is very large, the cleaning machine usually runs continuously. Each cleaning test is time-consuming, so the manual sampling detection is very inconvenient.

### SUMMARY OF THE INVENTION

In view of the above shortcomings, the primary object of the present invention is to provide a monitoring system for automatically monitoring a working fluid in a tank, comprising the tank, a sampling unit, a detecting agent tank, and a control module. The tank has a sampling opening. The sampling unit is connected to the sampling opening of the tank for obtaining a sample of the working fluid from the tank. The detecting agent tank is connected to the sampling unit for injecting a detecting agent to the sample. The control module includes a processing unit and a detector. The processing unit is electrically connected to the sampling unit for controlling the sampling unit to obtain the sample. The detector is configured to detect the sample of the working fluid and input a detection result to the processing unit.

The present invention may be the control module of the monitoring system.

Preferably, the control module includes a timer for controlling the sampling unit to obtain the sample periodically.

Preferably, the sampling unit includes a static mixer and a sampling tank. The static mixer is connected to the sampling opening of the tank and the detecting agent tank. The sample is discharged to the sampling tank after being mixed in the static mixer.

Preferably, the sampling unit has a sampling tank. A cleaning unit and a collection tank are connected to the sampling tank. The cleaning unit is electrically connected to the processing unit. After the detector obtains the detection result, the processing unit controls the cleaning unit to clean the sampling tank and discharges a waste liquid in the sampling tank to the collection tank.

Preferably, the cleaning unit includes a water cleaning machine and an air blower.

Preferably, the tank is located in a working machine. The working machine has a power switch electrically connected to the processing unit. When the detection result of the sample exceeds a setting value, the processing unit turns off the power switch of the working machine.

Preferably, the detector is a pH detector or an optical detector. The detection result is pH information or optical information. The processing unit converts the pH information or the optical information into concentration information.

According to the above technical features, the following effects can be achieved:

1. The working fluid can be automatically sampled (or periodically sampled) to automatically determine whether the working fluid is usable, so that no manual operation detection is required to reduce the workload of the medical staff.

2. When the detection result of the sample exceeds the setting value, the working fluid in the tank is determined to be unusable, and the power switch of the working machine of the tank is automatically turned off to prevent the unusable working fluid from being used continuously.

3. The invention is particularly suitable for monitoring medical equipment, such as an endoscope reprocessor, which can avoid the medical equipment from being not disinfected fully when the concentration of the working fluid (cleaning liquid) is insufficient; as a result, the viral bacteria of the previous patient are transmitted to the next patient to cause a cross-infection.

4. The detector may use an electric signal or an optical signal for detecting the concentration, temperature, pH value and the like of the working fluid to determine whether the working fluid is usable.

### BRIEF DESCRIPTION OF THE DRAWINGS

FIG. 1 is block diagram of the monitoring system of the present invention; and

FIG. 2 is an operation flowchart of the monitoring system of the present invention.

### DETAILED DESCRIPTION OF THE PREFERRED EMBODIMENTS

According to the above technical features, the main functions of the monitoring system and its control module of the present invention will be clearly shown in the following embodiments.

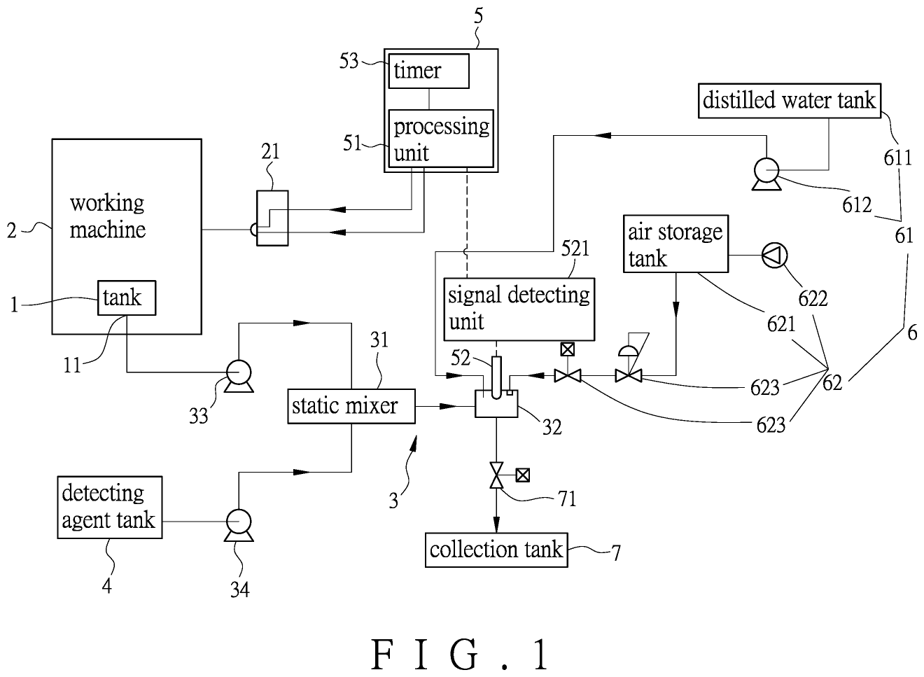

Referring to FIG. 1, the monitoring system in accordance with an embodiment of the present invention comprises a tank (1), a working machine (2), a sampling unit (3), a detecting agent tank (4), and a control module. (5), a cleaning unit (6), and a collection tank (7).

The tank (1) is disposed in the working machine (2), and the tank (1) has a sampling opening (11). The working machine (1) has a power switch (21) electrically connected to a processing unit (51) of the control module (5). The sampling unit (3) is connected to the sampling opening (11) of the tank (1) and the detecting agent tank (4). The sampling unit (3) includes a static mixer (31), a sampling tank (32), a first pump (33) disposed between the sampling opening (11) of the tank (1) and the static mixer (31), and a second pump (34) disposed between the detecting agent tank (4) and the static mixer (31). The control module (5) includes the processing unit (51) and a detector (52) that is electrically connected to the processing unit (51). The detector (52) has a signal detecting unit (521). The detector (52) corresponds to the sampling tank (32). The control module (5) has a timer (53). The processing unit (51) is electrically connected to the first pump (33) and the second pump (34) of the sampling unit (3) for controlling the opening and closing of the first pump (33) and the second pump (34). The cleaning unit (6) is connected to the sampling tank (32). The cleaning unit (6) includes a water cleaning machine (61) and an air blower (62). The water cleaning machine (61) has a distilled water tank (611) and a third pump (612) disposed between the sampling tank (32) and the distilled water tank (611). The processing unit (51) is electrically connected to the third pump (612) for controlling the opening and closing of the third pump (612). The air blower (62) has an air storage tank (621), a compressor (622) connected with the air storage tank (621), and a switching valve (623) disposed between the sampling tank (32) and the air storage tank (621). The processing unit (51) is electrically connected to the compressor (622) and the switching valve (623) for controlling the opening and closing of the compressor (622) and the switching valve (623). The collection tank (7) is connected to the sampling tank (32). A drain valve (71) is disposed between the collection tank (7) and the sampling tank (32). The processing unit (51) is electrically connected to the drain valve (71) for controlling the opening and closing of the drain valve (71).

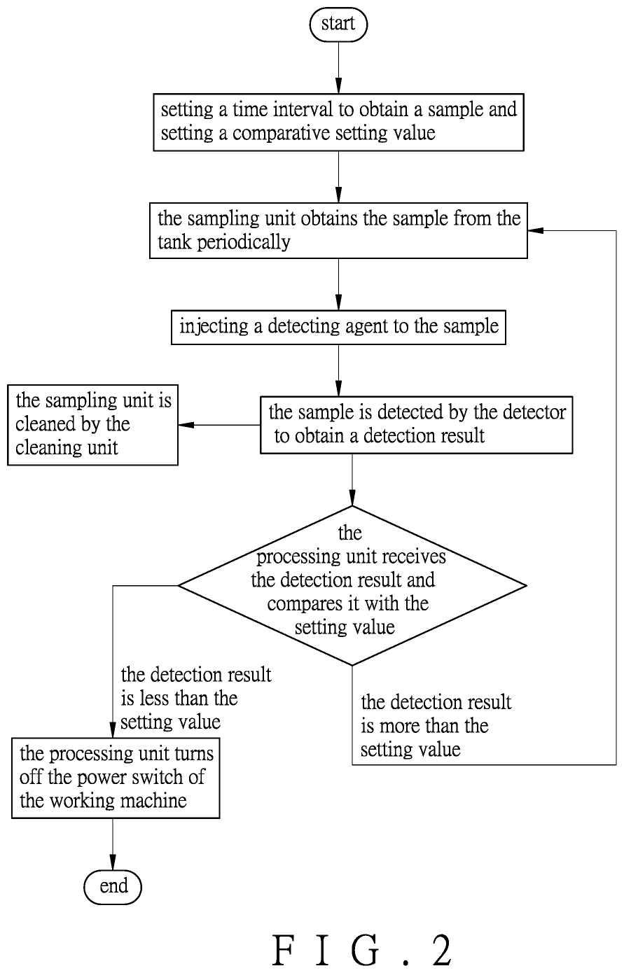

Referring to FIG. 1 and FIG. 2, in this embodiment, the working machine (2) takes an endoscope reprocessor as an example. A working fluid is poured into the tank (1). The working fluid is a cleaning fluid for cleaning endoscopes, such as o-phthalaldehyde or glutaraldehyde. When the working machine (2) is running, the time interval for taking a sample is first set by the timer (53), and then the processing unit (51) periodically and continuously controls the first pump (33) and the second pump (34) to be turned on according to the set time interval and extracts a sample of the working fluid from the tank (1) to the static mixer (31). The detecting agent in the detecting agent tank (4) is injected into the sample, and the sample is thoroughly mixed with the detecting agent in the static mixer (31) and then discharged into the sampling tank (32). The sample is detected by the detector (52), and then a detection result is input to the processing unit (51). The detector (52) may be, for example, a pH detector or an optical detector. The detection result is pH information or optical information. In this embodiment, in order to confirm whether the concentration of the cleaning fluid is sufficient, the processing unit (51) converts the pH information or the optical information into concentration information. The conversion formula is a well-known formula and will not be described hereinafter. For the processing unit (51) to determine whether the concentration of the cleaning liquid is sufficient, a setting value of the concentration of the cleaning liquid is set in the processing unit (51) in advance. The setting value is the lowest concentration value that the cleaning liquid is still usable. Therefore, when the detection result of the sample is less than the setting value, the processing unit (51) will turn off the power switch (21) of the working machine (2) to notify the operator to replace the cleaning liquid, preventing the unusable cleaning fluid from being used continually. When the endoscope after use cannot be completely disinfected, it is likely to have a risk of cross-infection. After the detector (52) obtains the detection result, the processing unit (52) controls the drain valve (71) to open and discharge the sample to the collection tank (7) and simultaneously controls the third pump (612) to open and extract the distilled water in the distilled water tank (611) to rinse the sampling tank (32). The processing unit (52) further controls the compressor (622) and the switching valve (623) to open, and the compressed air of the air storage tank (621) is blown to the sampling tank (32) to remove residual liquid, thereby ensuring that subsequent samples are not infected to achieve periodically continuous detections.

In addition to the aforementioned periodical detection, it is also possible to pre-detect whether the cleaning liquid is usable before the endoscope is to be cleaned each time. The detection, for example, adopts a current sensor to sense when the working machine (2) generates a current every time, and the sample is extracted for detection.

Through the automatic sampling (or periodically continuous sampling) to automatically determine whether the cleaning liquid is usable or not, no manual operation detection is required, and the workload of the medical staff is reduced. In addition to determine whether the cleaning liquid for endoscopes is usable, the present invention may be used to determine whether various industrial fluids are usable.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. A monitoring system for monitoring a working fluid in a tank, comprising:
the tank, having a sampling opening;
a sampling unit, connected to the sampling opening of the tank for obtaining a sample of the working fluid from the tank;
a detecting agent tank, connected to the sampling unit for injecting a detecting agent to the sample;
a control module, including a processing unit and a detector, the processing unit being electrically connected to the sampling unit for controlling the sampling unit to obtain the sample, the detector being configured to detect the sample of the working fluid and input a detection result to the processing unit.

2. The monitoring system as claimed in claim 1, wherein the control module includes a timer for controlling the sampling unit to obtain the sample periodically.

3. The monitoring system as claimed in claim 1, wherein the sampling unit includes a static mixer and a sampling tank, the static mixer is connected to the sampling opening of the tank and the detecting agent tank, and the sample is discharged to the sampling tank after being mixed in the static mixer.

4. The monitoring system as claimed in claim 1, wherein the sampling unit has a sampling tank; a cleaning unit and a collection tank are connected to the sampling tank, the cleaning unit is electrically connected to the processing unit, after the detector obtains the detection result, the processing unit controls the cleaning unit to clean the sampling tank and discharges a waste liquid in the sampling tank to the collection tank.

5. The monitoring system as claimed in claim 4, wherein the cleaning unit includes a water cleaning machine and an air blower.

6. The monitoring system as claimed in claim 1, wherein the tank is located in a working machine, the working machine has a power switch electrically connected to the processing unit, when the detection result of the sample exceeds a setting value, the processing unit turns off the power switch of the working machine.

7. The monitoring system as claimed in claim 1, wherein the detector is a pH detector or an optical detector, the detection result is pH information or optical information, and the processing unit converts the pH information or the optical information into concentration information.

8. A control module of a monitoring system for monitoring a working fluid in a tank, comprising:
a processing unit;
a detector, electrically connected to the processing unit, the detector being configured to detect a sample of the working fluid and input a detection result to the processing unit.

9. The control module of the monitoring system as claimed in claim 8, further comprising a timer for setting a time interval to obtain the sample periodically.

10. The control module of the monitoring system as claimed in claim 8, wherein the detector is a pH detector or an optical detector, the detection result is pH information or optical information, and the processing unit converts the pH information or the optical information into concentration information.
